(19) United States
(12) Patent Application Publication
(10) Pub. No.: US 2026/0083611 A1
(43) Pub. Date: Mar. 26, 2026

(54) OPERATING CHAIR FOR SUPPORTING AN OPERATOR

(71) Applicant: HELLSTERN MEDICAL GMBH, Wannweil (DE)

(72) Inventors: Sabrina HELLSTERN, Wannweil (DE); Claudia SODHA, Gechingen (DE); Alexander STROBEL, Haigerloch-Stetten (DE); Harald RAGER, Bisingen-Wessingen (DE)

(21) Appl. No.: 19/111,635

(22) PCT Filed: Sep. 11, 2023
(86) PCT No.: PCT/EP2023/074891

§ 371 Date: Mar. 13, 2025

(30) Foreign Application Priority Data
Sep. 15, 2022 (DE) 10 2022 123 601.3

(51) Int. Cl. A61G 15/08

(52) U.S. Cl. CPC A61G 15/08

(57) ABSTRACT

The operating chair according to the invention with a tipping protection device serves to support the operator (surgeon) with the largest possible range of motion. The operating chair has a platform that can be placed on the floor, from which a column with a pivoting device extends at least substantially vertically, the pivoting device being designed to pivot the column about at least one axis relative to the platform The operating chair also has a seat attached to the column on which the surgeon can sit. The column can also have a height adjustment device with which the height of the seat above the platform can be adjusted. The tipping protection device is configured to alter the standing area of the operating chair.

### CROSS REFERENCE TO RELATED APPLICATIONS

The present application is a National Stage of PCT Application No. PCT/EP2023/074891 filed on Sep. 11, 2023, which claims priority to German Patent Application No. 10 2022 123 601.3 filed on Sep. 15, 2022, the contents each of which are incorporated herein by reference thereto.

### TECHNICAL FIELD

The present disclosure relates to an operating chair (surgical chair) for supporting an operator (surgeon), particularly during a medical operation, such as a surgical procedure.

### BACKGROUND

Operating chairs (surgical chairs) are generally known and serve to support the operator (surgeon) who normally stands at the operating table, in such a way that the sometimes long-lasting operations in a forced posture or operations (surgeries) with special demands on the operator's fine motor skills can be performed by the operator (surgeon) with little or no fatigue and, ideally, in a reliable and high-quality manner.

DE 10 2020 103 861 B3, for example, discloses an operation chair (surgical chair) that serves to support an operator (surgeon). This operating chair has a seat supported by a height-adjustable column, the column standing on a joint device that defines two tilt axes for a column and to which a locking device is assigned in order to lock manually set tilt positions so that the column maintains the desired tilt position. The column extends from a mobile, accessible platform that defines the standing area of the operating chair (surgical chair). The dimensions of the platform are such that the platform has the largest possible standing area in order to enable the largest possible tilt range in which the column can be safely tilted. The stability of the operating chair (surgical chair) is of utmost importance, since the operator (surgeon) holds medical instruments such as scalpels in his hands during operation (surgery), which can pose a risk to the health of the patient and the surgeon if the operating chair (surgical chair) is unstable. However, the space available for the operating chair in the surgical area, for example in the operating room, is quite limited, which means that the standing area defined by the platform is kept as compact as possible. However, this limits the potential range of motion, for example for the tilt positions and/or height positions of the column, in which the operating chair (surgical chair) can offer the operator (surgeon) secure support.

### BRIEF SUMMARY

Based on this, the object of the present disclosure is to provide an improved operating chair for an operator (surgeon) that offers a large range of motion for the operator (surgeon), in which he is held securely by the operating chair.

Disclosed is an operating chair for supporting an operator including: a mobile platform from which a column with a pivoting device extends at least substantially vertically, wherein the pivoting device is configured to pivot the column about at least one axis relative to the platform; a seat for the operator attached to the column; and a tipping protection device configured to change a standing area of the operating chair.

The operating chair according to the present disclosure comprises a mobile platform, which can be placed on the floor, from which a column with a pivoting device extends at least substantially vertically, the pivoting device being configured to pivot the column about at least one axis relative to the platform. The operating chair also has a seat attached to the column for the surgeon to sit on. The column preferably also has a height adjustment device with which the height of the seat above the platform is adjustable. The operating chair also has a tipping protection device configured to alter the standing area of the operating chair.

The tipping protection device enables the standing area of the operating chair to be increased in a targeted manner, particularly when the operating chair is positioned in a desired location in the operating area. This makes it possible for the pivot device to be used to safely set larger tilt positions for the column without the operating chair becoming unstable and tipping over.

The key feature of the operating chair according to the present disclosure is that the standing area of the operating chair can be increased and decreased as required by the tipping protection device. For example, the standing area of the operating chair can be increased when the operating chair is in the desired position in the operating area (surgical area). For transport, the standing area of the operating chair can be reduced again, for example, so that the operating chair can be brought into the most compact state possible, in which it can be transported as easily as possible through doorways and/or corridors in the hospital.

The standing area of the operating chair is understood to mean in particular an area that is projected vertically onto the floor from the outer points of the operating chair.

Preferably, the operating chair also has an upper body holding device for supporting the upper body of the operator (surgeon). The upper body holding device is particularly attached to the upper end of the column. Alternatively, the upper body holding device may be attached to the platform. The upper body holding device not only supports the operator (surgeon) by enabling him to sit on the seat of the operating chair, but also holds the upper body of the operator.

In the simplest case, the upper body holding device can be a support backrest. However, it is preferred that the upper body holding device is configured to hold back the upper body of the operator with a traction device, wherein the operator wears a harness that can be detachably coupled to a free end of the traction device. For example, the upper body holding device has a support column at an adjustable height where a free end of the traction device is provided.

This support column is preferably located behind the operator sitting on the operating chair. The other end of the traction device can be wound up or unwound by an electrically operated winch, for example, so that the length of the traction device, which is provided outside the upper body holding device is adjustable. A support angle for the operator's upper body can be adjusted via the length of the traction means. The support angle is preferably continuously adjustable. The support column of the upper body holding device can be attached to an upper end of the column, for example.

In particular, the tipping protection device has at least one support unit with a support that can be extended and retracted along an extension axis, whereby the standing area of the operating chair can be changed. Preferably, the tipping protection device has two or more extendable and retractable support units such that the standing area can be changed. The tipping protection device is preferably housed within the platform, with the extendable and retractable supports extending beyond the edge of the platform when they are in an extended state. The support units can, for example, be arranged in such a way that they can be extended sideways. This increases the standing area of the operating chair in the lateral direction, so that a larger range of motion can be released for the pivoting device of the column when the supports of the support unit are extended compared with when the supports of the support unit are retracted. If the column is additionally configured to be height-adjustable through a height-adjustment device, a larger range of motion for the height-adjustment device can be released when the supports of the support unit are extended compared with when the supports of the support unit are retracted. It is preferred that the supports be extended without the use of an electric motor, for example, by spring-force.

Preferably, a support foot is attached to an end region of the support. The support foot can ensure that the support rests securely on the floor and at the same time serve as an actuating element to extend the support.

In particular, the end region of the support in the extended state is located farther away from a side surface of the platform than in the retracted state. By extending the support, the standing area of the operating chair can thus be enlarged. It is preferred that the end regions of the supports in the extended state define a standing area of the operating chair that is larger than a standing area of the operating chair in the retracted state.

The support is particularly arranged such that the extension axis has a predetermined depth angle relative to a surface of the platform. The extension axis of the support points towards the floor in the direction of extension, so that the support foot can be made smaller. Alternatively, the support can be extended in particular at least substantially parallel to the surface of the platform.

Preferably, the support foot of the support in the extended state is spaced from the floor. This ensures, on the one hand, that the support can be fully extended without the support foot prematurely touching the floor. Furthermore, the operating chair can continue to be moved without the supports dragging on the floor when the operating chair is moved. The platform can also have a platform height adjustment device with which the platform can be raised and lowered so that the height of the platform above the floor can be changed.

Preferably, the support foot is shaped in the form of a plate with an actuating surface and a supporting surface, the actuating surface being larger than the supporting surface. The actuating surface is preferably flush with the side surface of the platform when the support is retracted. The actuating surface is configured to be as large as possible so that the support units can be extended and retracted with the foot. The supports can be extended, for example, in the retracted state, for example, by a person pressing the foot, for example, the tip of the foot against the actuating surface, so that the supports are moved from the retracted state to the extended state, for example driven by a spring.

In particular, the support foot is in a first position when the support is in a retracted state and in a second position when the support is in the extended state of the support. Preferably, the first position is located closer to the center of gravity of the operating chair than the second position.

Preferably, the support foot can be moved manually from the first position to a third position, wherein the support unit is configured to move the support foot to the second position when the support foot has been manually moved to the third position. For this purpose, the support unit can include, for example, a spring with which the support is pushed towards the second position. The first, second and third positions of the support foot are preferably arranged on the extension axis. The first position is located between the second position and the third position.

It is preferred that the column also has a height adjustment device that is configured to adjust the height of the column and thus the seat above the platform.

In a particularly preferred embodiment, the operating chair further has a control device configured to predetermine a first range of motion to the pivoting device and/or the height adjustment device and/or the upper body holding device within which they can be moved.

Preferably, the support unit has at least one sensor element configured to at least detect whether the support is in the extended state. Additionally, the sensor element may also be configured to detect whether the support is in the retracted state. Furthermore, the support unit may have additional sensor elements that also enable to detect whether the support is in the retracted state.

In particular, the sensor element is communicatively connected to the control device, wherein the control device is configured to predetermine a second range of motion to the pivoting device and/or the height adjustment device and/or the upper body holding device when the support is in the extended state, wherein a second range of motion is larger than the first range of motion.

Alternatively, the control device may be configured to deactivate the movability of individual or multiple adjustment devices, such as the pivoting device and/or the height adjustment device and/or the upper body holding device, when the support unit is in the retracted state. For example, the pivoting device can be switched to an immobile mode by the control device when the tipping protection device is in the retracted state.

### BRIEF DESCRIPTION OF THE DRAWINGS

Further details of advantageous further developments or details of the present disclosure arise from the drawings, the description and the dependent claims. The Figures show:

FIG. 1 a perspective view of the operating chair for supporting the operator according to the present disclosure.

FIG. 2 a schematic cross-section of the tipping protection device in the retracted state.

FIG. 3 a schematic cross-section of the tipping protection device in the actuated state.

FIG. 4 a schematic cross-section of the tipping protection device in the extended state, and

FIG. 5 a schematic top view of the operating chair.

### DETAILED DESCRIPTION

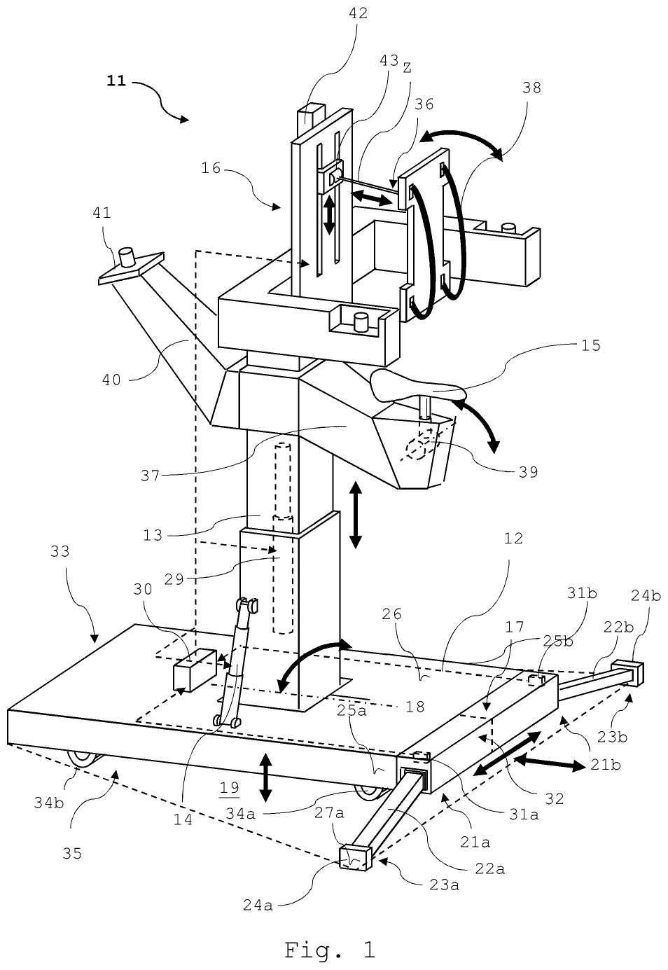

FIG. 1 shows a perspective view of an embodiment of the operating chair 11. The operating chair 11 comprises a mobile platform 12, which can be moved in at least two directions using a driving device 35. The driving device 35 can have at least three, preferably four, electrically driven wheels 34a, 34b, only two of which are shown in FIG. 1. The platform also has a platform height adjustment device with which the height of the platform above the floor can be changed.

The operating chair 11 has a front side 32 and a rear side 33, between which the main body of the platform 12 extends. The operating chair can be arranged for operation such that the front side 32 of the platform 12 faces the surgical area. The platform 12 has at least a substantially flat surface 26, as well as two side surfaces 25a and 25b.

column 13 protrudes from the surface 26 of the platform 12 at least substantially vertically. The column 13 can be pivoted laterally about an axis 18 by means of a pivoting device 14. The column can also be equipped with another pivoting device, which makes it possible, for example, to pivot the column in a forward-backward direction about a further axis, which is arranged, for example, orthogonal to the first axis.

The column 13 also houses a height adjustment device 29, with which the height of the column 13 and thus of the seat 15 attached to the column 13 above the platform 12 can be adjusted.

A first longitudinal beam 37 is arranged on an upper side of the column 13, which extends from the column 13 to the front 32 of the operating chair 11. The seat 15, on which the surgeon can sit during the operation, is attached to one end of the first longitudinal beam 37. The seat 15 can be tilted forwards and backwards by means of a tilting device 39. At the upper end of the column 13, a second longitudinal beam 40 is also arranged, which extends from the column 13 towards the rear 33 of the platform 12. At the end of the second longitudinal beam 40, an operating unit 41 is arranged, with which, for example, the driving device 35 and/or the platform height adjustment device can be controlled.

Above the column 13 extends the upper body holding device 16 with which the upper body of the operator (surgeon) can be held at a certain inclination angle. The upper body holding device 16 has a support column 42 on which an adjustable deflection roller 43 is arranged. The deflection roller 43 guides the traction device Z of the upper body holding device 16. At a free end 36 of the traction device Z, a harness 38 worn by the operator can be detachably coupled to the upper body holding device. The inclination angle at which the operator's upper body is held can be adjusted via the length of the traction device Z.

The operating chair 11 has a tipping protection device 17 configured to change a standing area 19 of the operating chair 11. In the example shown in FIG. 1, the tipping protection device 17 has a first support unit 21a and a second support unit 21b. The first and second support units 21a and 21b are housed in the front side 32 of the operating chair 11 within the platform 12. The support units 21a and 21b each have an extendable and retractable support 22a and 22b, which are movable along extension axis 20a and 20b. The extension axis 20a and 20b extend laterally from the platform 12.

At an end region 23a and 23b of the supports 22a and 22b, support feet 24a and 24b are attached to the supports 22a and 22b.

Additionally, the tipping protection device 17 may have further support units, which may be housed, for example, on the rear side 33 of the operating chair 11 within the platform 12.

The support units 21a and 21b shown in FIG. 1 are in an extended state in which the supports 22a and 22b are extended. The standing area 19 of the operating chair 11 is larger than when one or both of the support units 21a and 21b are in a retracted state. The support units 21a and 21b can be brought into an extended or a retracted state independently of one another.

The standing area 19 is spanned by the corners of the platform at the rear side 33 and the support feet 24a and 24b. When the support units 21a and 21b are in the retracted state, the standing area 19 of the operating chair 11 is spanned by the corners of the platform 12.

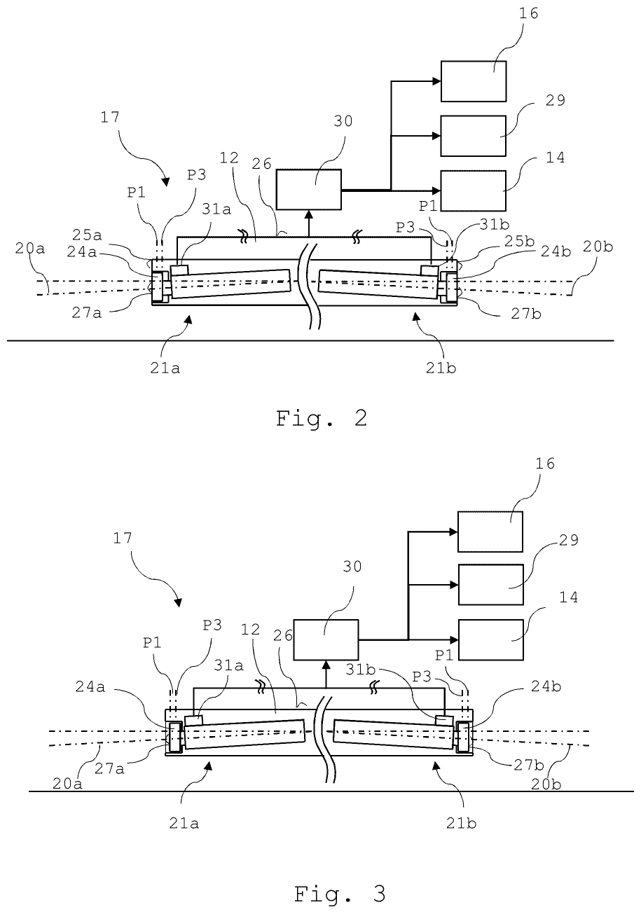

FIG. 2 shows a schematic cross-section of the tipping protection device 17 with both support units 21a and 21b from FIG. 1. The extendable and retractable supports 22a and 22b are both shown in a retracted state in FIG. 2. The support feet 24a and 24b are arranged in a first position P1 in FIG. 2. The support feet 24a and 24b each have an actuating surface 27a and 27b and a supporting surface 28a and 28b wherein the actuating surface 27a and 27b are flush with the side surfaces 25a, 25b of the platform 12 in a first position P1. The supports 22a and 22b are held in the first position P1 in the retracted state by springs. To extend the supports 22a and 22b, the support feet 24a and 24b can be manually pressed against a spring force along the extension axis 20a and 20b into a third position P3.

FIG. 3 shows this schematic cross-section of the tipping protection device 17 from FIG. 2, but the support feet 24a and 24b are arranged in the third position in FIG. 3. The support units 21a and 21b are configured to push the supports 22a and 22b along the extension axis 20a and 20b into a second position P2 when the support feet have been brought into the third position P3.

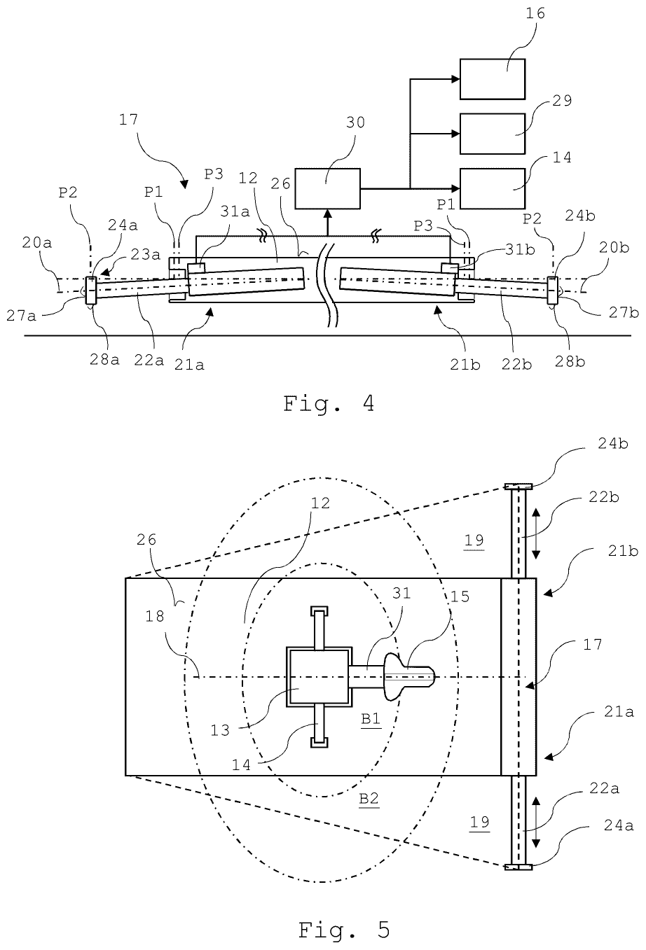

FIG. 4 shows the tipping protection device 17 from FIGS. 2 and 3 with the support feet 24a, 24b arranged in the second position P2. The supports 22a, 22b are shown in an extended state in FIG. 4. From the extended state, the supports 22a, 22b can be brought back into the retracted state by pushing the support feet 24a, 24b into the third position P3. When the first position P1 is passed by the support feet 24a, 24b, the supports 22a, 22b are held in the first position P1 by a locking device, with the spring force of the spring continuing in the extension of the supports 22a, 22b. However, the locking device prevents the support feet 24a, 24b from passing the first position P1.

In the example of the tipping protection device 17 shown in FIGS. 2 to 4, the extension axis 20a and 20b are arranged at an angle relative to the surface 26 of the platform 12. The support units 21a and 21b shown also each have at least one sensor element 31a and 31b, which are configured to detect whether the respective support units 21a and 21b are in an extended or retracted state. The sensor elements 31a and 31b are communicatively connected to a control device 30. The control device 30 can be communicatively connected to the pivoting device 14, the height-adjustment device 29 and the upper body holding device 16.

The control device 30 is configured to predetermine a first range of motion B1 and a second range of motion B2 for the pivoting device 14 and/or the height-adjustment device 29 and/or the upper body holding device 16, in which the pivoting device 14 and/or the height adjustment device 29 and/or the upper body holding device 16 are moveable. The control device 30 can predetermine the first range of motion when at least one of the supports 22a and 22b is in a retracted state and the second range of motion B2 when both supports 20a and 20b are in the extended state. The second range of motion B2 is larger than the first range of motion B1. Alternatively, for example, the pivoting device 14 can be put into an immovable mode by the control device 30 when both supports 22a and 22b are in a retracted state.

FIG. 5 shows a schematic top view of the operating chair 11 from FIGS. 1 to 4. The extendable and retractable supports 22a, 22b are extended in the example shown in FIG. 5, whereby enlarging the standing area 19 of the operating chair 11 beyond the boundaries of the platform 12. FIG. 5 also shows the first range of motion B1 and the second range of motion B2, with the second movement area B2 completely enclosing the first movement area B1.

The operating chair 11 described so far works as follows:

The operating chair 11 can be transported to a desired position with the support units 21a, 21b of the tipping protection device 17 in a retracted state. The standing area 19 of the operating chair 11 is defined by the platform 12. In a retracted state, at least one adjustment device, such as the pivoting device 14 of the operating chair 11, is only movable within a limited range of motion B1. For example, the pivoting device 14 can also be put in an immobile mode by the control device 30 when the tipping protection device 17 is in a retracted state. When the operating chair 11 is placed in a desired location, the support units 21a, 21b of the tipping protection device 17 can be brought into an extended state. The extension of the supports 22a, 22b can be triggered by pressing with the foot against the actuating surface 27a, 27b, whereupon the supports 22a, 22b are pushed outwards in the direction of the extension axis 28, 20b, for example by a spring. The support units 21a, 21b can be extended independently of one another.

The operating chair 11 according to the present disclosure with the tipping protection device 17 serves to support the operator (surgeon) in the largest possible range of motion B2. The operating chair 11 has a platform 12 that can be set up on the floor, from which a column 13 with a pivoting device 14 extends at least substantially vertically, wherein the pivoting device 14 is configured to pivot the column 13 about at least one axis 18 relative to the platform 12. The operating chair 11 also has a seat 15 attached to the column 13, on which the operator (surgeon) can sit. The column 13 can also have a height adjustment device 29 with which the height of the seat 15 above the platform 12 is adjustable. The tipping protection device 17 is configured to alter a standing area 19 of the operating chair 18.

### REFERENCE SIGNS

11 operating chair
12 platform
13 column
14 pivoting device
15 seat
16 upper body holding device
17 tipping protection device
18 axis
19 standing area of the operating chair
20a, b extension axis
21a, b support unit
22a, b extendable and retractable support
23a, b end region of the support
24a, b support foot
25a, b side surface of the platform
26 surface of the platform
27a, b actuating surface
28a, b supporting surface
29 column height adjustment device
30 control device
31a, b sensor element
32 front side of the operating chair
33 rear side of the operating chair
34a, b wheel of the driving device
35 driving device
36 free end of the traction device
37 first longitudinal beam
38 harness worn by the surgeon
39 tilting device
40 second longitudinal beam
41 Operating Unit
42 support column of the upper body holding device
B1, first range of motion
B2 second range of motion
P1 first position
P2 second position
P3 third position

1. An operating chair for supporting an operator comprising:
a mobile platform from which a column with a pivoting device extends at least substantially vertically, wherein the pivoting device is configured to pivot the column about at least one axis relative to the platform;
a seat for the operator attached to the column; and
a tipping protection device configured to change a standing area of the operating chair.

2. The operating chair according to claim 1, wherein the operating chair further comprises an upper body holding device to support an upper body of the operator, the upper body holding device being preferably attached to the column.

3. The operating chair according to claim 1, wherein the tipping protection device comprises at least one support unit with a support that is extendable and retractable along an extension axis.

4. The operating chair according to claim 3, wherein a support foot is attached in an end region of the support.

5. The operating chair according to claim 4, wherein the end region of the support in an extended state is arranged further away from a side surface of the platform than in a retracted state.

6. The operating chair according to claim 4, wherein the end regions of the supports in an extended state defines a standing area of the operating chair that is larger than a standing area of the operating chair in a retracted state.

7. The operating chair 11 according to claim 4, wherein the support is arranged such that the extension axis has a predetermined depth angle relative to a surface of the platform.

8. The operating chair according to claim 4, wherein the support is extendable at least substantially parallel to a surface of the platform.

9. The operating chair according to claim 4, wherein the support foot of the support is arranged at a distance from a floor in an extended state.

10. The operating chair according to claim 4, wherein the support foot is plate-shaped with an actuating surface and a supporting surface, wherein the actuating surface is larger than the supporting surface.

11. The operating chair according to claim 10, wherein the actuating surface in a retracted state of the support is at least substantially flush with a side surface of the platform.

12. The operating chair according to claim 4, wherein the support foot is in a first position in a retracted state of the support and is in a second position in an extended state of the support.

13. The operating chair according to claim 12, wherein the support foot is manually moveable from the first position to a third position, wherein the at least one support unit is configured to bring the support foot into the second position when the support foot has been manually brought into the third position.

14. The operating chair according to claim 1, wherein the column has a height adjustment device configured to adjust a height of the column above the platform.

15. The operating chair according to claim 14, wherein the operating chair further comprises a control device configured to predetermine a first range of motion for the pivoting device and/or the height adjustment device in which they are moveable.

16. The operating chair according to claim 3, wherein the at least one support unit comprises at least one sensor element configured to detect whether the support is in an extended state.

17. The operating chair according to claim 15, wherein the tipping protection device comprises at least one support unit with a support that is extendable and retractable along an extension axis, the at least one support unit comprises at least one sensor element configured to detect whether the support is in an extended state and the at least one sensor element is communicatively connected to the control device, wherein the control device is configured to predetermine a second range of motion for the pivoting device and/or the height adjustment device when the support is in the extended state, wherein the second range of motion is larger than the first range of motion.

18. The operating chair according to claim 2, wherein the tipping protection device comprises at least one support unit with a support that is extendable and retractable along an extension axis.

19. The operating chair according to claim 18, wherein a support foot is attached in an end region of the support.

20. The operating chair according to claim 19, wherein the end region of the support in an extended state is arranged further away from a side surface of the platform than in a retracted state.
